(19) United States
(12) Patent Application Publication
(10) Pub. No.: US 2026/0080981 A1
(43) Pub. Date: Mar. 19, 2026

(54) METHOD FOR DESIGNING COMPOSITION OF LAYERED STRUCTURE OF LYC COMPOUND AND RECORDING MEDIUM COMPRISING SAME

(71) Applicants: Lotte Energy Materials Corporation, Jeollabuk-do (KR); SEOUL NATIONAL UNIVERSITY R&DB FOUNDATION, Seoul (KR)

(72) Inventors: Jongho RYU, Jeollabuk-do (KR); Joohyeon NOH, Jeollanam-do (KR); Takrae HA, Jeollabuk-do (KR); Jeonghyun LEE, Jeollabuk-do (KR); Kisuk KANG, Seoul (KR); Joonwoo PARK, Jeollabuk-do (KR); Seungju YU, Seoul (KR)

(21) Appl. No.: 18/932,960

(22) Filed: Oct. 31, 2024

(30) Foreign Application Priority Data
Sep. 13, 2024 (KR) 10-2024-0125708

(51) Int. Cl. G16C 60/00; G16C 20/30

(52) U.S. Cl. CPC G16C 60/00; G16C 20/30

(57) ABSTRACT

Disclosed is a method for designing the composition of a layered structure of Li₃YCl₆ compound exhibiting enhanced ionic conductivity, which provides a method for designing the composition of a lithium yttrium halide solid electrolyte with a hexagonal close-packed structure, the method being executed by a processor, the method including: calculating possible diffusion paths for lithium ions to migrate an adjacent octahedral site in the a-b plane in each consecutive layer constituting a unit cell with a hexagonal close-packed structure; calculating the activation barrier energy for lithium ion diffusion for each of the calculated diffusion paths; and determining the occupancy of yttrium within the unit cell to form a percolation state where diffusion paths with a low calculated activation barrier energy are connected within the unit cell.

### CROSS-REFERENCE TO RELATED APPLICATION

This application is based on and claims priority under 35 U.S.C. 119 to Korean Patent Application No. 10-2024-0125708, filed on Sep. 13, 2024, in the Korean Intellectual Property Office, the disclosure of which is herein incorporated by reference in its entirety.

### FIELD

The present disclosure relates to a method for designing the composition of a solid electrolyte containing a halide and, more specifically, to a method for designing the composition of a layered structure of Li₃YCl₆ compound exhibiting enhanced ionic conductivity and a computer-readable recording medium for implementing the method.

### BACKGROUND

Semi-solid-state or all-solid-state batteries with solid electrolytes partially or completely replacing liquid electrolytes are attracting increasing interest and research.

However, the solid electrolytes are required to have excellent interfacial characteristics between electrolyte layers containing the same and active material layers and, furthermore, superior contact characteristics between solid electrolytes and active material particles, along with high lithium ionic conductivity comparable to those of liquid electrolytes.

Various solid electrolytes meeting these requirements have been continuously searched for since before, and for example, sulfide-based solid electrolytes, oxide-based solid electrolytes, and halide-based solid electrolytes have been proposed.

Sulfide-based solid electrolytes have the advantages of facilitating the induction of close contact between solid electrolytes and active material particles due to their excellent flexibility compared with oxide-based solid electrolytes and having relatively excellent lithium ionic conductivity, but have the disadvantages of being less stable when exposed to moisture or oxygen in the air and presenting challenges in the battery manufacturing process. Additionally, oxide-based solid electrolytes have the disadvantages of having difficulty in securing excellent contact properties between solid electrolytes and active materials and showing insufficient ionic conductivity.

In recent years, halide-based solid electrolytes exhibiting excellent stability and a predetermined level of ionic conductivity have been proposed to solve the disadvantages of the sulfide- or oxide-based solid electrolytes.

Most of the halide-based solid electrolytes have a layered structure, a rock-salt structure, or a similar structure. Out of these, particularly, hcp-Li₃YCl₆-based solid electrolytes have various compositions with multiple components, such as Zr and lanthanides, being substituted or added.

These halide-based solid electrolytes are known to exhibit high levels of ionic conductivity due to mechanochemical synthesis, but the mechanisms thereof have not been revealed, so that it has not yet reached a level where the ionic conductivity of halide-based solid electrolytes can be controlled or halide solid electrolytes with high ionic conductivity are freely designed.

Therefore, there is a need to develop design rules for the composition of layered structures of halides, such as a Li₃YCl₆ compound.

### PRIOR ART DOCUMENTS

### Patent Documents

(Patent Document 1) (1) KR 10-2023-0092885 A
(Patent Document 2) (2) JP 2023-519758 A

### SUMMARY

The present disclosure has been made to solve the problems of conventional art, and an aspect of the present disclosure is to provide a composition design method for optimizing the ionic conductivity of a Li₃YCl₆ compound.

Another aspect of the present disclosure is to provide a composition design method for optimizing the occupancy of metal ions within a layer in a Li₃YCl₆ compound with a hexagonal close-packed structure.

Still another aspect of the present disclosure is to provide a computer-readable storage medium for implementing the above-described method for designing the composition of a halide.

In accordance with an aspect of the present disclosure, there is provided a method for designing the composition of a lithium yttrium halide solid electrolyte with a hexagonal close-packed structure, the method being executed by a processor, the method including: calculating possible diffusion paths for lithium ions to migrate an adjacent octahedral site in the a-b plane in each consecutive layer constituting a unit cell with a hexagonal close-packed structure; calculating the activation barrier energy for lithium ion diffusion for each of the calculated diffusion paths; and determining the occupancy of yttrium within the unit cell to form a percolation state where diffusion paths with a low calculated activation barrier energy are connected within the unit cell.

The percolation state may include a Y-1 ordering with one yttrium atom and a Y-free ordering with no yttrium atom in terms of the number of yttrium atoms per unit cell in the ab-plane.

The method calculating of the activation barrier energy may be performed by first-principles calculation-based nudged elastic band (NEB) simulation.

The diffusion paths may pass through a tetrahedral site within the unit cell.

The diffusion paths with a low calculated activation barrier energy may include a T_{Y}T_{V} path where one of two octahedral sites adjacent to the tetrahedral site is occupied by yttrium and the other is vacant.

The diffusion paths with a low calculated activation barrier energy may include a T_{V}T_{V} path where both of two octahedral sites adjacent to the tetrahedral site are vacant.

The T_{Y}T_{V} path has a lower activation barrier energy than the T_{V}T_{V} path.

The determining of the occupancy may include: calculating the interlayer distance between adjacent layers within the unit cell according to the occupancy of yttrium; calculating the occupancy of yttrium at which the interlayer distance according to the occupancy of yttrium becomes saturated; and establishing the occupancy of yttrium at which the interlayer instance becomes saturated, as a lower limit of the occupancy of yttrium.

The calculating of the interlayer distance according to the occupancy of yttrium may be performed by first-principles calculation simulation.

The determining of the occupancy may include establishing the maximum value of the occupancy of yttrium for formation of the percolation state as an upper limit of the occupancy of yttrium.

The method may further include: after the determining of the occupancy, calculating the composition of the lithium yttrium halide on the basis of the occupancy of yttrium.

In accordance with another aspect of the present disclosure, there is provided a computer-readable storage medium storing instructions configured to cause, when executed by at least one processor, to implement the above-described predetermined operations by the at least one processor.

According to the present disclosure, a method for designing a novel composition of a halide can be provided to optimize the ionic conductivity of a halide-based Li₃YCl₆ compound.

Furthermore, according to the present disclosure, a method for designing the composition of a halide can be provided to optimize the occupancy of metal ions within a layer in a Li₃YCl₆ compound with a hexagonal close-packed structure.

### BRIEF DESCRIPTION OF THE DRAWINGS

The above and other aspects, features and advantages of the present disclosure will be more apparent from the following detailed description taken in conjunction with the accompanying drawings.

FIG. 1A shows the crystalline structure of hcp-Li₃YCl₆, and

FIGS. 1B, 1C and 1D illustrate possible arrangement models for different types of metal arrangements in the crystalline structure of hcp-Li₃YCl₆.

FIG. 2A exemplifies the ordering of metal ions for each arrangement model, and

FIGS. 2B, 2C, 2D and 2E illustrate the lithium diffusion path at each site.

FIG. 3A is a graph showing the calculation results of the activation barrier according to the ordering of metal ions, and

FIGS. 3B, 3C and 3D illustrate a difference in lithium conductivity path according to the ordering of metal ions.

FIGS. 4A and 4B illustrate the design rule of the halide composition according to an embodiment of the present disclosure.

FIGS. 5A, 5B and 5C illustrate the occupancy of metal ions in each layer and the ionic conductivity path according to the occupancy in a halide composition with a hexagonal close-packed structure.

FIG. 6 is a graph obtained by calculating and plotting a target composition according to an embodiment of the present disclosure and the occupancy in each layer within a unit cell with a hexagonal close-packed structure in the target composition.

FIG. 7 is a table summarizing the compositions of conventional literature plotted in FIG. 6 and sources thereof.

FIG. 8 is a graph showing the X-ray diffraction analysis results of a halide sample manufactured according to an embodiment of the present disclosure.

FIG. 9 is a schematic diagram showing a device to which a composition design method is applicable according to an embodiment of the present disclosure.

### DETAILED DESCRIPTION

It is to be noted that in the following description, only the parts necessary for understanding exemplary embodiments of the present disclosure will be described, and descriptions of the other parts will be omitted so as not to deviate from the gist of the present disclosure.

The terms and words used in this description and the appended claims are not to be interpreted in common or lexical meaning but, based on the principle that an inventor can adequately define the meanings of terms to best describe the disclosure, to be interpreted in the meaning and concept conforming to the technical concept of the present disclosure. The features described in exemplary embodiments and drawings shown herein are for merely illustrating one of the most preferable exemplary embodiments but are not intended to represent the technical idea of the present disclosure, and thus the present disclosure may cover various equivalents and modifications which can substitute for the exemplary embodiments at the time of filing the present application.

Herein, a compound defined by mentioning its constituent elements may include compounds where some of the mentioned elements are substituted or additional elements besides the mentioned ones are included. Herein, when referring to a compound by constituent elements, for example, lithium yttrium chloride (or LYC) does not exclusively refer to a ternary compound consisting of lithium, yttrium, and chlorine, but also includes quaternary or higher order compounds that contain one or more other elements substituting some of the metal ions or further contain other interstitial elements within the crystal structure.

Hereinafter, a layered structure of halide solid electrolyte, such as an LYC compound, and a composition design procedure therefor will be described with reference to drawings.

A. Crystalline structure of hcp-Li₃YCl₆

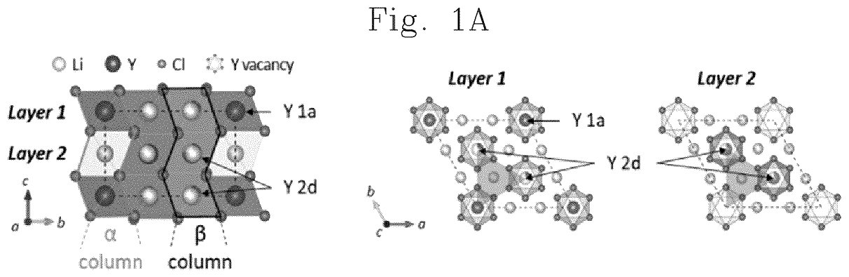

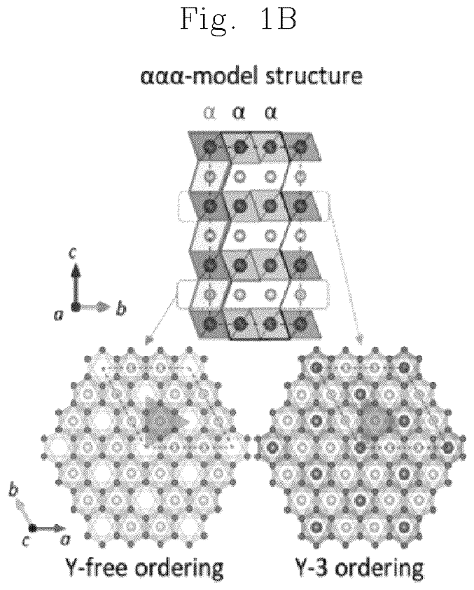

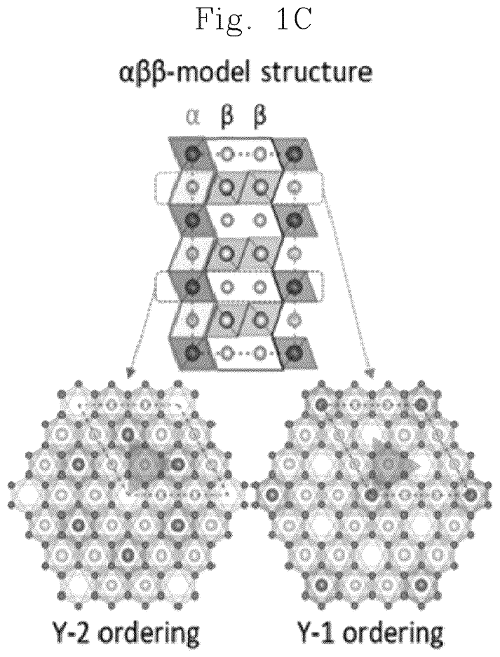

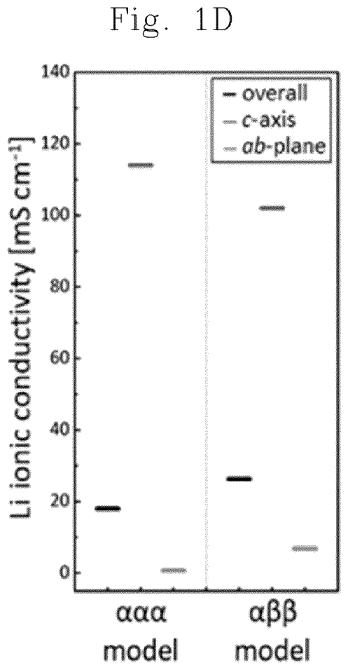

FIG. 1A shows the crystalline structure of hcp-Li₃YCl₆ (herein, hcp-Li₃YCl₆ and compounds based on this structure are collectively referred to as hop-LYC), and FIGS. 1B and 1C show two possible arrangement models, that is, ααα model and αββ model, for different types of metal arrangements in the hcp-LYC structure.

Referring to FIG. 1A, in the exemplary structure of hcp-LYC, the two layers, Layer 1 and Layer 2, are consecutively arranged, and in each layer, halogen ions, such as Cl⁻ ions, form a hexagonal close-packed structure, and lithium ions and metal ions are ordered in octahedral sites formed by halogen ions.

As shown in FIG. 1A, there are a total of two sites where the metal ions can occupy in each layer, of which one is the 1a site located at the vertex of the unit cell and the other is the 2d site located at the center within each layer.

### B. Layered Structure of Hcp-LYC and Lithium Diffusion Path Modeling

To understand the change tendency of ionic conductivity according to the number and arrangement of metals, two models may be established according to the position of metal ions in the 2d site. Between the two models, one contains the ααα-layer where metal ions are present in both the 2d site and the 1a site as shown in FIG. 1B, and the other contains the αββ-layer where ions in the 2d site and the 1a site are separated by a layer as shown in FIG. 1C. The latter structure may be a most stable arrangement in the hcp-LCY structure.

The ionic conductivity of the overall structure and the ab-plane/c-axis conductivity for the two models were calculated and are shown in FIG. 1D. The ionic conductivity was calculated using ab initio molecular dynamics (AIMD) simulations, with the Vienna ab-initio simulation package (VASP) software used as software.

Referring to FIG. 1D, the αββ model had a higher conductivity.

Noticeably, the overall ionic conductivity of the ααα model is lower than that of the αββ model since, despite having a higher c-axis diffusion rate compared with the αββ model, the ααα model shows a lower diffusion rate in the ab-plane compared with the αββ model. This suggests that the ab-plane diffusion determines the overall ionic conductivity in the corresponding structure. Therefore, the effect of the metal arrangement on the diffusion in the ab-plane needs to be examined.

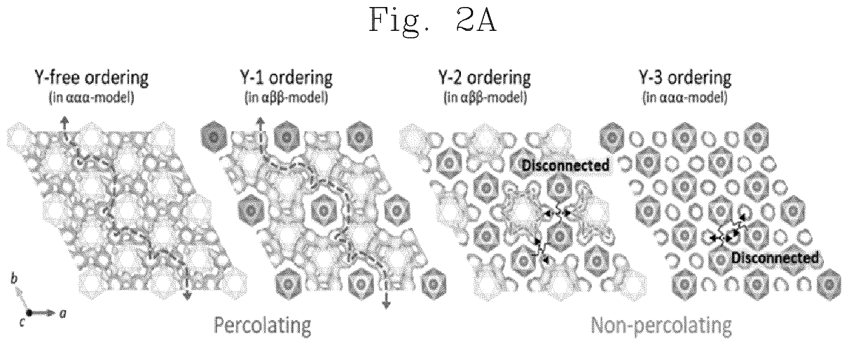

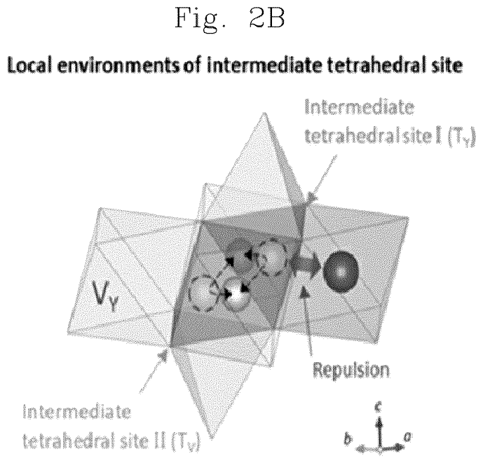

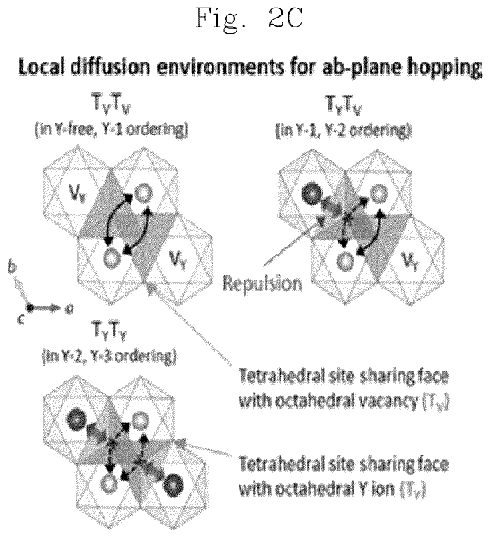

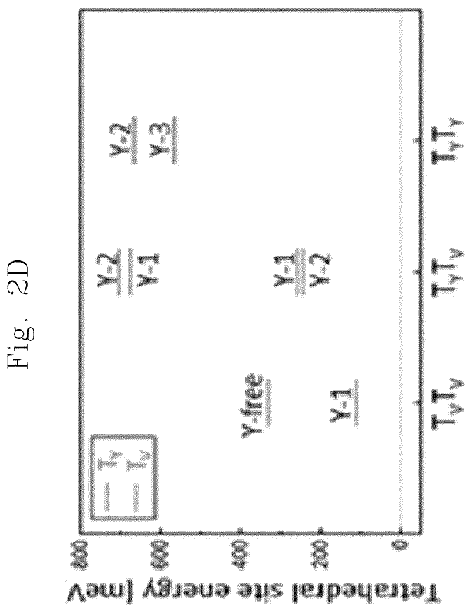

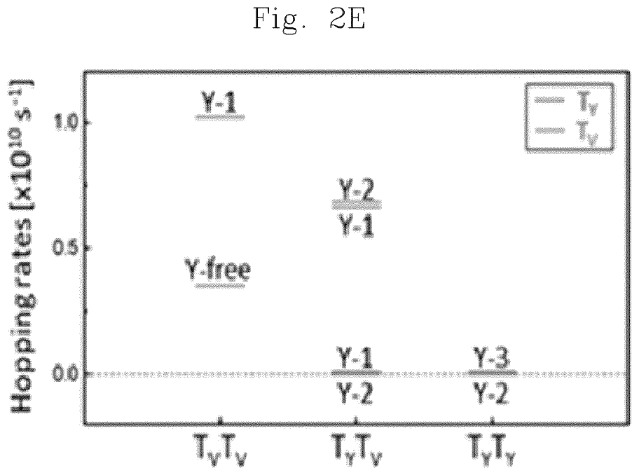

FIG. 2A exemplifies the ordering of metal ions in each model. As shown in FIG. 2A, Y-free ordering and Y-3 ordering are possible in the ααα model, and Y-1 ordering and Y-2 ordering are possible in the αββ model. Particularly, the number following Y indicates the number of metal atoms per unit cell in the ab-plane.

When the layers of each model are individually separated and examined for sites at which Li may be present in the ab-plane, Y-free ordering without no metal ions and with one metal ion overall can create an environment that entirely enables Li percolation, and Y-2 ordering and Y-3 ordering may create an environment where Li is isolated among metal ions, resulting in a difficulty in ion diffusion.

FIG. 2B illustrates the lithium diffusion path at each site.

Referring to FIG. 2B, lithium ions, for migration, need to pass through an intermediate tetrahedral site that is face-shared with an octahedral site that can be occupied by a metal. If the diffusion path when a metal ion is present in the octahedral site is denoted by T_{Y}, and the diffusion path when the octahedral site is vacant is denoted by T_{V}, T_{Y} corresponds to an environment where lithium ions can hardly pass through due to strong repulsion of the metal ion, whereas T_{V} with no metal ion corresponds to an environment where lithium ions can easily pass through the tetrahedral site.

As described later, in the present disclosure, various physical quantities are calculated on the basis of the first-principles calculations based on quantum chemistry. In the present disclosure, the ionic conductivity, hopping rate, activation battier energy, interlayer distance, lithium probability density, specific site energy, and the like are examples of calculable physical quantities.

### C. Activation Barrier Energy Calculation According to Lithium Diffusion Path

The activation barrier energy required for lithium ions to pass through a tetrahedral site is calculated depending on whether the metal ion occupies an octahedral site. In the present disclosure, the activation barrier energy may be calculated on the basis of the first-principles calculation.

In the present disclosure, the ab-initio molecular dynamic (AIMD) and nudged elastic band (NEB) techniques may be used for the activation barrier energy calculations.

The first-principles calculation refers to a method for calculating the properties of a material, by a computational technique for solving the Schrödinger equation to describe the state of a material on the basis of quantum chemistry, without using any empirical quantities, considering a material basic constant, such as Planck's constant or electron mass, and the interactions between the nucleus and electrons and the interactions between electrons within an atom. These calculations are also called ab-initio calculations due to the non-use of empirical quantities.

In the present disclosure, the migration behavior of lithium ions at a predetermined temperature may be observed using ab-initio molecular dynamics (AIMD), and through the observation results, the ionic conductivity of lithium ions and the frequency of hopping of lithium may be measured. Especially, through the procedure of finding the most stable state when lithium migrates, lithium diffusion paths and the activation energy required to pass through each of the paths can be calculated. Particularly, nudged elastic band (NEB) simulations may be used to analyze lithium diffusion characteristics.

The present disclosure may be implemented by interworking with commercial software to perform the first-principles calculation. In the present disclosure, for instance, software modules including AIMD and NEB utilizing the Vienna ab-initio Simulation Package (VASP) may be reasonably used to perform the first-principles calculations, for first-principle calculations.

The commercial software VASP was used to calculate the activation barrier energy through NEB simulations, and the hopping rates in the AIMD simulation results were analyzed by a MATLAB program.

FIGS. 2D and 2E are graphs showing the simulation results. Referring the drawings, the presence of metal ions on both sides of the lithium migration path (i.e., T_{Y}T_{Y}) resulted in a low hopping rate since the migration of lithium was difficult due to a very high energy required for passage, while the presence of a metal ion at only one side (that is, T_{V}T_{Y}) enables the migration of lithium ions through T_{V} with low energy but not T_{Y} with high energy. The absence of metal ions at both sides allowed for the migration of lithium ions through both the paths.

Interestingly, the ion diffusion is faster in T_{V}T_{Y} or T_{Y}T_{V} where one metal ion is present on the lithium path than T_{V}T_{V} where no metal ion is present on the lithium path. This can be explained as follows.

When comparing a Y-free ordering layer with no metal ion present within one layer with a Y-1 ordering layer with one metal ion present within one layer, the large difference with respect to the lithium conductivity is that the sectional area (i.e., the interlayer distance) of the most critical path for plane diffusion depends on the number of metal ions.

To investigate the effect of interlayer distance on diffusion, the T_{V}T_{V} activation barrier was calculated by arbitrarily changing the interlayer distance for each situation. The activation barrier was calculated by the nudged elastic band (NEB) simulation, and VASP was used as software.

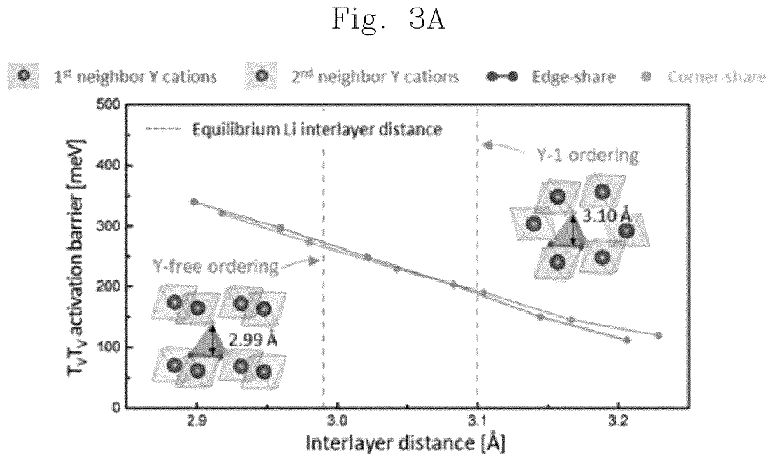

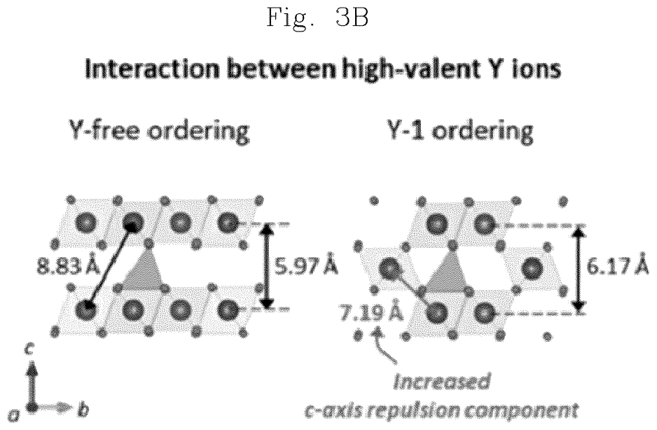

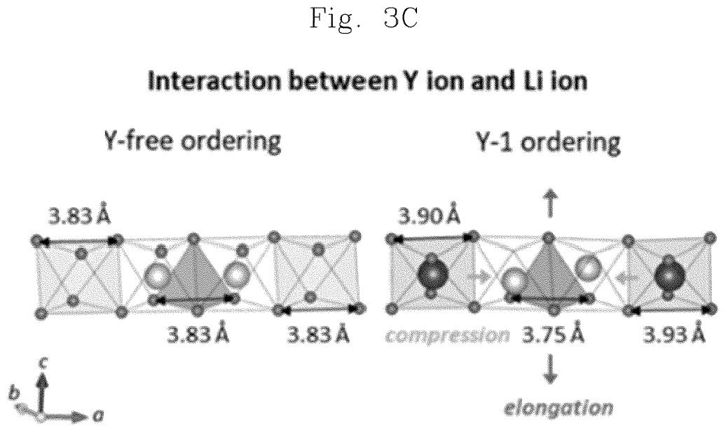

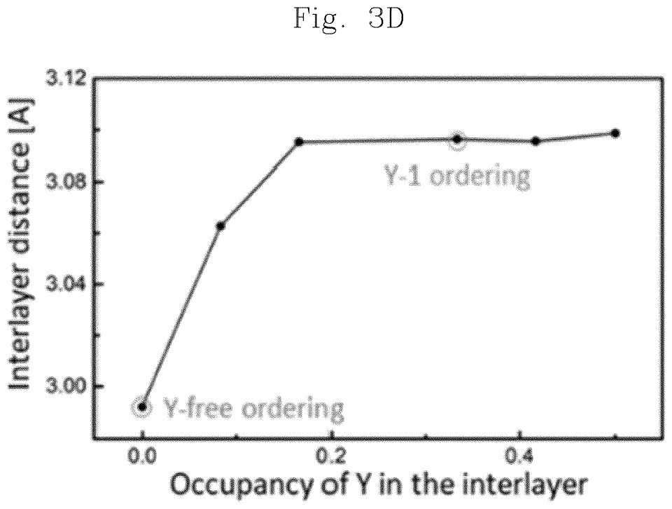

FIG. 3A is a graph showing the calculation results of the T_{V}T_{V} activation barrier. It can be confirmed that the T_{V}T_{V} activation barrier changes in the same manner according to the interlayer distance, regardless of the presence or absence of metal ions in the layer. It can also be confirmed that the activation barrier in diffusion decreases with increasing interlayer distance.

### D. Effect of Occupancy of Yttrium on Interlayer Distance

Meanwhile, the results of calculating the effect of the occupancy of Y on the interlayer distance are shown in FIG. 3D. The corresponding interlayer distances were calculated by analyzing the simulation results of first-principles calculations. VASP was used as software.

Referring to FIG. 3D, the interlayer distance increased until the occupancy of Y reached 0.166, and then became saturated.

In other words, it can be seen that the change in diffusion degree between the Y-free ordering with no metal ion and the Y-1 ordering is attributed to a difference in this interlayer distance, with the path of lithium ions widening to some extent when the number of metal ions is 0.166 or greater.

Additionally, as shown in FIG. 3C, it can be calculated that the presence of Y on the lithium diffusion path results in a distortion in intermediate sites, and this elongation of the tetrahedral site also had an effect on the expansion of the interlayer distance.

FIG. 3B shows that the distance between neighboring Y ions becomes markedly shorter in the Y-1 layer (7.19 Å) than in the Y-free layer (8.83 Å), leading to a stronger c-axis repulsion component between Y ions in adjacent planes, which contributes to an increased interlayer space from 5.97 to 6.17 Å.

### E. Calculation of Occupancy for Optimal Percolation State

As described above, the diffusion in the ab-plane can be fast when the environment within a layer enables the percolation of lithium ions and the interlayer distance is sufficiently wide. Meanwhile, a small number of metal ions within a layer is advantageous in percolation, but an appropriate number of metal ions is required to widen the interlayer distance.

The range satisfying the two conditions may be specified as follows. The occupancy of metal ions within each layer preferably has at least a predetermined lower limit value to sufficiently ensure the interlayer distance and at most a predetermined upper limit value to provide percolation within each layer. Preferably, the lower limit value may be 0.166 and the upper limit value may be 0.444 in the present disclosure.

In the present disclosure, the upper limit value may be determined as follows.

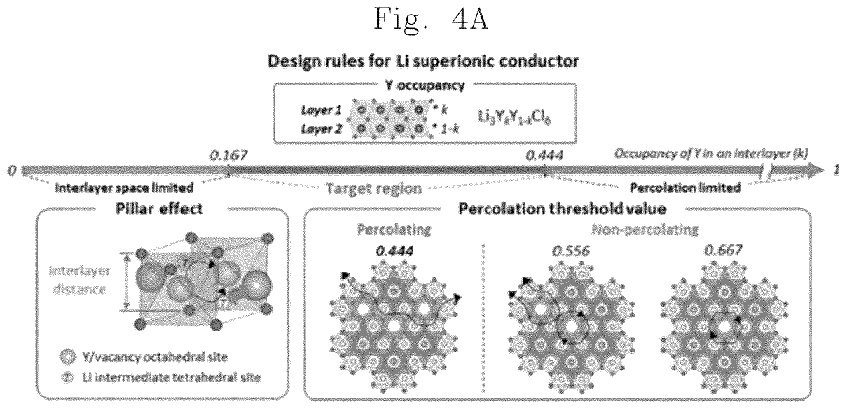

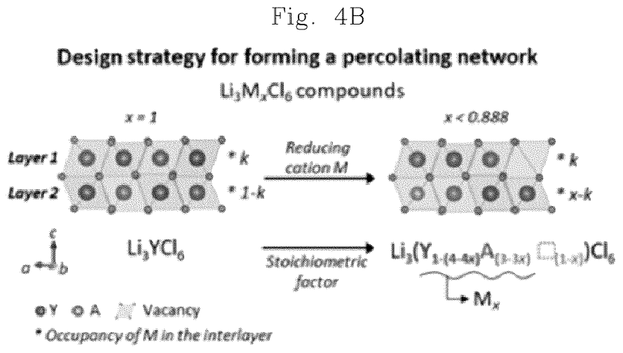

Referring to FIG. 4A, when a hexagonal unit cell is established based on metal ions, the central metal ion needs to be vacant and two metal ions at the vertices of the unit cell need to be vacant in order to allow the percolation of lithium ions, that is, in order for all lithium ions in the hexagonal unit cell to be in the T_{V}T_{Y} or T_{V}T_{V} state. Lithium ions cannot pass through the unit cell at occupancies greater than this level.

FIG. 4A illustrates and shows the calculation design of threshold values for percolation. A hexagonal unit cell contains one central metal ion site and six vertex sites, and the total number of metal ion sites within one unit cell is three since the vertex sites are shared by two adjacent unit cells. The maximum number of metal ions in the unit cell in the threshold state allowing percolation where the central metal ion is vacant and the two vertex metal ions are vacant in the unit cell is (4*1/3), and the occupancy of metal ions may be calculated as ((4*1/3)/3=4/9=0.444).

Based on these results, the design rule for a state that enables lithium percolation may be determined.

As described above, the hcp-LYC structure is composed of two layers of consecutive arrangements, and in a composition where the metal-to-anion ratio is 1:6, a total of six metal ion sites and a total of three metal ions are present in the two layers. In a composition with a metal-to-anion ratio of 1:6, all the metal ion sites may be occupied by metal ions (e.g., Y-3 ordering), and in such a case, the occupancy may be expressed as 1. Whereas, all metal ion sites in another consecutive layer may be vacant (e.g., Y-free ordering), and in such a case, the occupancy may be expressed as 0. Therefore, each layer in the hcp-LYC structure may have an occupancy of 0 to 1, and if the occupancy of one layer is k, the occupancy of the consecutive layer may be expressed as 1-k. In other words, the sum of the occupancies of two consecutive layers must be 1.

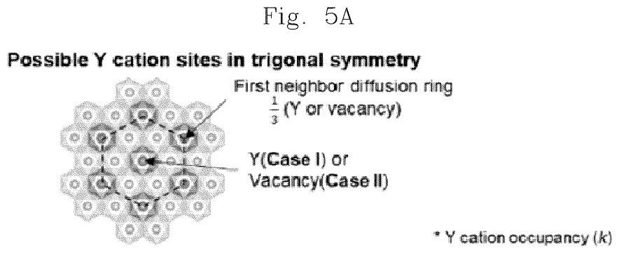

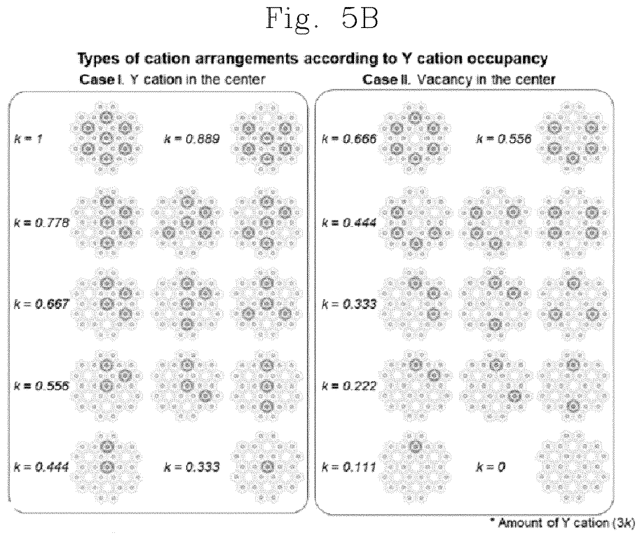

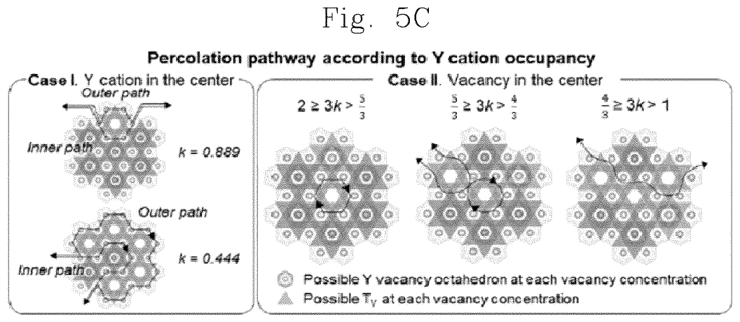

FIG. 5A shows Y ion sites in the hexagonal unit cell. As shown, one central Y ion site and six vertex Y ion sites are present.

In FIG. 5B, CASE I indicates the occupancy k of Y ions depending on the occupation of each vertex when the Y ion site in the center is occupied by Y ion, and CASE II indicates the occupancy k of Y ions depending on the occupation of each vertex when the Y ion site in the center is vacant.

FIG. 5C shows the Li percolation path according to the occupancy k of Y ions.

As described above, the threshold occupancy of one layer allowing ion percolation is 0.444, and if one of two adjacent layers has an occupancy of metal ions of k=0.444, the occupancy of the other consecutive layer, 1-k, must have a value of 0.556 (=1-0.444). In such a case, the other layer is necessarily a non-percolating layer due to the number of metal ions exceeding the threshold value.

In order to achieve high ionic conductivity of the hcp-LYC solid electrolyte, the sum of the occupancies of two adjacent layers needs to be designed to be less than 0.444*2=0.888 since the maximum value of the occupancy of metal ions within the structure is 0.444.

### F. Designing Hcp-LYC Composition

Based on these findings, the inventors of the present disclosure designed a composition capable of achieving high ionic conductivity without changing the number of Li within a unit cell while maintaining the sum of the metal occupancies of two consecutive layers at 0.888 or less.

In other words, the present disclosure provides a hcp-LYC-based solid electrolyte composition, in which the number of metal ions included is decreased to achieve an occupancy of 0.444 or less in individual layers and a sum of occupancies of 0.888 or less in two layers.

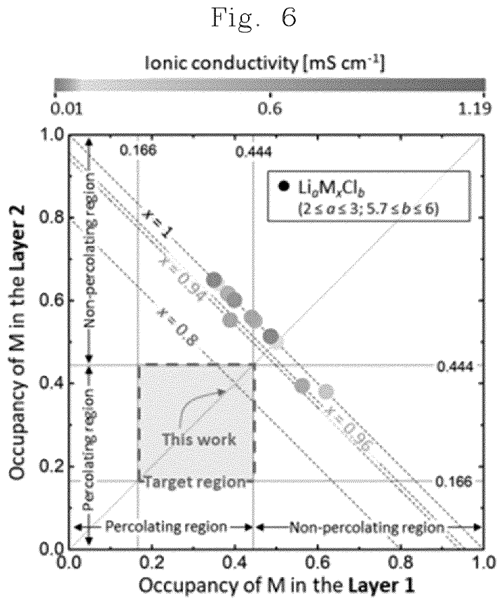

FIG. 6 is a graph obtained by calculating and plotting the occupancies of Layer 1 and Layer 2 in the unit cell for a target composition of the present disclosure and conventional compositions reported in the prior literature. Referring to FIG. 6, the conventional compositions are out of the target region of the present disclosure, with relatively high metal contents. In the formula LiₐMₓCl_{b} shown in FIG. 6, M represents a trivalent or tetravalent ion, such as Y, Ho, Er, or Zr, constituting the hcp-trigonal structure.

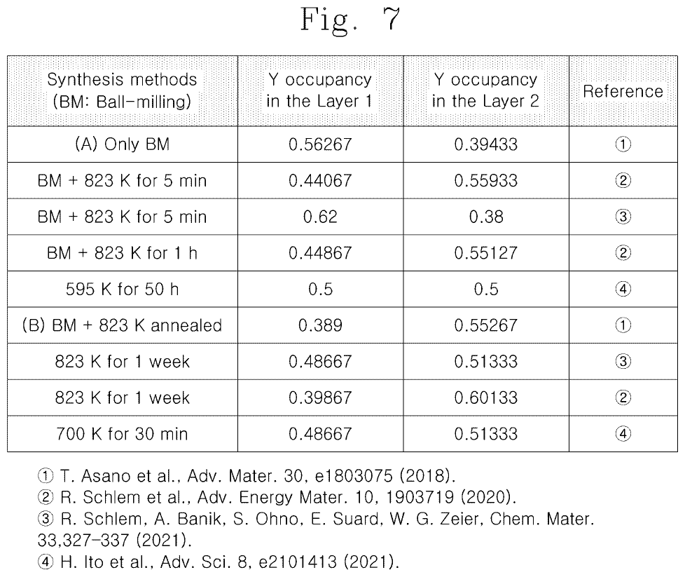

FIG. 7 is a table summarizing the compositions from conventional literature and the literature sources, as plotted in FIG. 6.

The composition of the present disclosure may be expressed by the following composition formula.

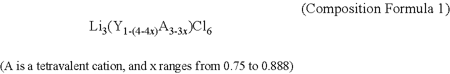

In the present disclosure, A includes at least one element selected from the group consisting of Ti, Zr, Hf, and Rf.

In the present disclosure, the range of the x value may be calculated as follows.

Herein, 1−(4−4x) simplifies 4x−3, and 4x−3≥0 needs to be satisfied, which leads to x≥0.75. In Composition Formula 1, the number of atoms, 4x−3+3−3x, simplifies x, and the value of x is constrained to a minimum of 0.333 and a maximum of 0.888, which correspond to twice the percolation threshold in each layer, 0.167 to 0.444. Therefore, the x value satisfying the two conditions becomes 0.75≤x≤0.888, and a preferable LYC composition in the present disclosure can be optimized within the above-described x value range.

As in Composition Formula 1, the composition formula that reflects vacancies created by substituting the trivalent ion with a higher tetravalent ion (A⁴⁺) is as follows.

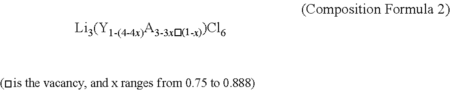

Considering that the charge balance is maintained by, among the number of substituted tetravalent ions, creating vacancies corresponding to some of the substituted ions and decreasing the number of lithium ions corresponding to other of the substituted ions, the composition formula of the present disclosure may be expressed more generally as follows:

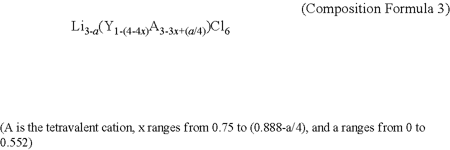

The composition formula that reflects on the created vacancies is as follows.

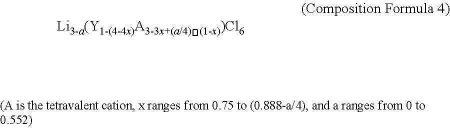

Composition Formulas 3 and 4, when a representing the reduction ratio of lithium ions is 0, becomes the same as Composition Formulas 1 and 2, respectively.

Hereinafter, actual experimental examples of the compositions of some solid electrolytes selected according to the above-described composition formulas of the present disclosure will be described.

### Experimental Example 1

To obtain compositions having the composition formulas on Table 1 below, LiCl, YCl₃, and ZrCl₄ were weighed according to the molar ratio of Li, Y, Zr, and Cl in the composition formulas, and then the compositions were synthesized by mechanochemical synthesis using a ball mill. As for mechanochemical synthesis conditions for the corresponding synthesis, synthesis was conducted using the Pulverisette 7PL equipment from Fritsch under 800 to 900 rpm, with 10 g of 10 mm ZrO₂ balls and 15 g of 5 mm ZrO₂ balls at a ball to powder ratio 25:1, through a total of 12 steps for 4 hours, with each step involving 15 minutes of milling and 5 minutes of rest.

The composition formulas of this experimental example correspond to the composition formula LiₐMₓCl_{b} in FIG. 6 where x representing the molar ratio of metal ions (M) is 0.8 to 0.888.

To measure the ionic conductivity of each of the synthesized compositions, a symmetrical cell with a SUS/solid electrolyte/SUS structure capable of preventing the deposition and desorption of ions in the electrolyte was used. The solid electrolyte was pelletized by compression under 254 MPa, and AC impedance measurements were conducted using VMP3 by Biologic at a frequency of 3 MHz to 100 MHz under a pressure of 75 MPa.

The ionic conductivity of each composition is shown in Table 1.

TABLE 1

Ionic conductivity (mS/cm)

| Sample | Composition Formula | 800 rpm | 900 rpm |
|---|---|---|---|
| #1 | Li₃Zr_{0.75}Cl₆ | 0.236 | 0.233 |
| #2 | Li₃Y_{0.1}Zr_{0.675}Cl₆ | 0.450 | |
| #3 | Li₃Y_{0.2}Zr_{0.6}Cl₆ | 1.19 | 1.19 |
| #4 | Li₃Y_{0.3}Zr_{0.525}Cl₆ | 0.690 | |
| #5 | Li₃Y_{0.4}Zr_{0.45}Cl₆ | 0.822 | |
| #6 | Li₃Y_{0.5}Zr_{0.375}Cl₆ | 0.818 | |
| #7 | Li₃Y_{0.552}Zr_{0.336}Cl₆ | 1.012 | 1.368 |

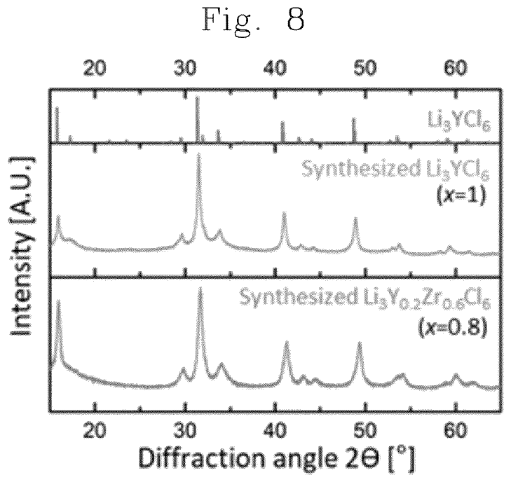

FIG. 8 is a graph showing the results of X-ray diffraction analysis for Sample #3. For comparison, XRD data for Li₃YCl₆ (i.e. x=1) is also plotted. FIG. 8 confirmed that Sample #3 had the same structure as the comparative example, Li₃YCl₆. In addition, the incorporation of Zr⁴⁺ shifted the diffraction pattern to the right, and this result indicates that the addition of Zr⁴⁺, which has a small ionic radius and induces the creation of vacancies, reduced the lattice constant.

### Experimental Example 2

To obtain compositions of the composition formulas on Table 2 below, starting materials were weighed according to the molar ratio of Li, Y, Zr, and Cl in the composition formulas, and then the compositions were synthesized. The composition formulas of this experimental example correspond to the compositional formula of LiₐMₓCl_{b} in FIG. 6 where x representing the molar ratio of metal ions (M) is 0.9 or larger.

The ionic conductivity of each of the synthesized compositions is shown in Table 2.

TABLE 2

| | | Ionic conductivity (mS/cm) |
|---|---|---|
| Sample | Composition Formula | 800 rpm |
| #8 | Li₃Y_{0.6}Zr_{0.3}Cl₆ | 0.594 |
| #9 | Li₃Y_{0.7}Zr_{0.22}Cl₆ | 0.506 |
| #10 | Li₃Y_{0.8}Zr_{0.15}Cl₆ | 0.720 |

### Experimental Example 3

To obtain compositions satisfying the composition formulas on Table 3 where the ratio of metal ions was 0.8, starting materials were weighed according to the molar ratio of Li, Y, Zr, and Cl in the composition formulas, and then the compositions were synthesized. The value of “a” in Composition Formula 3 was calculated and shown in the table.

The ionic conductivity of each of the synthesized compositions is shown in Table 3.

TABLE 3

| | | | Ionic conductivity (mS/cm) | |
|---|---|---|---|---|
| Sample | Composition Formula | a | 800 rpm | 900 rpm |
| #11 | Li₃Y_{0.2}Zr_{0.6}Cl₆ | 0 | 1.19 | 1.19 |
| #12 | Li_{2.9}Y_{0.1}Zr_{0.7}Cl₆ | 0.1 | 0.310 | 0.451 |
| #13 | Li_{2.8}Zr_{0.8}Cl₆ | 0.2 | 0.236 | 0.214 |

### Experimental Example 4

To obtain compositions satisfying the composition formulas on Table 4 where the ratio of metal ions was 0.85, starting materials were weighed according to the molar ratio of Li, Y, Zr, and Cl in the composition formulas, and then the compositions were synthesized. The value of “a” in Composition Formula 3 was calculated and shown in the table.

The ionic conductivity of each of the synthesized compositions is shown in Table 4.

TABLE 4

| | | | Ionic conductivity (mS/cm) | |
|---|---|---|---|---|
| Sample | Composition Formula | a | 800 rpm | 900 rpm |
| #14 | Li₃Y_{0.4}Zr_{0.45}Cl₆ | 0 | 0.776 | 0.487 |
| #15 | Li_{2.9}Y_{0.3}Zr_{0.55}Cl₆ | 0.1 | 1.203 | 0.708 |
| #16 | Li_{2.8}Y_{0.2}Zr_{0.65}Cl₆ | 0.2 | 0.867 | 0.959 |
| #17 | Li_{2.7}Y_{0.3}Zr_{0.75}Cl₆ | 0.3 | 0.967 | 0.665 |
| #18 | Li_{2.6}Zr_{0.85}Cl₆ | 0.4 | 0.408 | 0.463 |

### Experimental Example 5

To obtain compositions satisfying the composition formulas on Table 5 where the ratio of metal ions was 0.88, starting materials were weighed according to the molar ratio of Li, Y, Zr, and Cl in the composition formulas, and then the compositions were synthesized.

The ionic conductivity of each of the synthesized compositions is shown in Table 5.

TABLE 5

| | | | Ionic conductivity (mS/cm) | |
|---|---|---|---|---|
| Sample | Composition Formula | a | 800 rpm | 900 rpm |
| #19 | Li_{2.9}Y_{0.45}Zr_{0.436}Cl₆ | 0.1 | 1.137 | 1.366 |
| #20 | Li_{2.8}Y_{0.35}Zr_{0.536}Cl₆ | 0.2 | 1.401 | 1.403 |
| #21 | Li_{2.7}Y_{0.25}Zr_{0.636}Cl₆ | 0.3 | 0.915 | 0.613 |
| #22 | Li_{2.6}Y_{0.15}Zr_{0.736}Cl₆ | 0.4 | 0.999 | 0.324 |
| #23 | Li_{2.5}Y_{0.05}Zr_{0.836}Cl₆ | 0.5 | 0.692 | |
| #24 | Li_{2.448}Zr_{0.888}Cl₆ | 0.552 | 0.465 | 0.302 |

G. Devices to which Present Inventive Design Method is Applicable

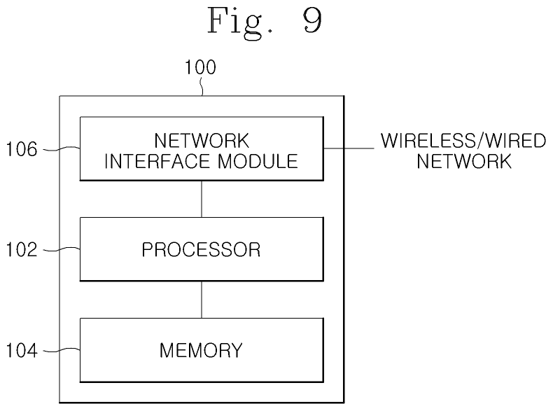

FIG. 9 exemplifies a device 100 to which the design method of the present disclosure is applicable. The device 100 may be configured to process data and information according to the composition design method of the present disclosure. The device 100 may be a user device, but is not limited thereto, and may be a server device providing a composition design service. For example, the device 100 to which the method of the present disclosure is applicable may include computer devices such as desktop computers and workstations, mobile terminals such as smart phones, portable devices such as laptop computers. As another example, the device 100 to which the present disclosure is applicable may be included, as a part of an application specific integrated circuit (ASIC) implemented in a system on chip (SoC) form.

A memory 104 may store programs for processing and controlling a processor 102, and may store data and information used in the present disclosure, control information necessary for data and information processing according to the present disclosure, temporary data created during data and information processing, and the like. The memory 104 may be implemented as a storage device, such as read only memory (ROM), random access memory (RAM), erasable programmable read only memory (EPROM), electrically erasable programmable read-only memory (EEPROM), flash memory, static RAM (SRAM), hard disk drive (HDD), or solid state drive (SSD).

One or more processors 102 control the operations of each module in the device 100. In particular, the processor 102 may perform various control functions for performing the proposed method of the present disclosure. The processor 102 may also be called a controller, a microcontroller, a microprocessor, a microcomputer, or the like. The proposed method of the present disclosure may be implemented by hardware, firmware, software, or a combination thereof. When the present disclosure is implemented using hardware, the processor 102 may be provided with an application specific integrated circuit (ASIC), a digital signal processor (DSP), a digital signal processing device (DSPD), a programmable logic device (PLD), a field programmable gate array (FPGA), and the like, which are configured to perform the present disclosure. Meanwhile, when the proposed method of the present disclosure is implemented using firmware or software, the firmware or software may include instructions related to modules, procedures, or functions that perform functions or operations necessary to implement the proposed method of the present disclosure. When the instructions are stored in the memory 104 or stored in a computer readable recording medium (not shown) separate from the memory 104 to be executed by the processor 102, the device 100 may be configured to implement the proposed method of the present disclosure.

Additionally, the device 100 may include a network interface module (NIM) 106. The network interface module 106 is operatively connected to the processor 102 during operation, and the processor 102 may control the network interface module 106 to transmit or receive wireless/wired signals that carry information and/or data, signals, messages, and the like, through a wireless/wired network. The network interface module 106 may support various communication standards, such as IEEE 802 series, 3GPP LTE(-A), and 3GPP 5G, and may transmit and receive control information and/or data signals according to the communication standards. The network interface module 106 may be implemented outside the device 100 as needed.

The embodiments described hereinabove are combinations of elements and features of the present disclosure in predetermined forms. Each element or feature should be considered selective unless otherwise explicitly stated. Each element or feature may be carried out without being combined with other elements or features. Some elements and/or features may be combined with each other to configure the exemplary embodiments of the present disclosure. The order of operations described in the exemplary embodiments of the present disclosure may be changed. Some elements or features of one exemplary embodiment may be included in another exemplary embodiment, or may be replaced by corresponding elements or features of another exemplary embodiment. It is apparent that some claims referring to specific claims may be combined with other claims referring to claims other than the specific claims to configure embodiments of the present disclosure or may be included as new claims by amendments after filing.

Meanwhile, the examples described in the present specification and drawings are merely the representation of specific examples to aid the understanding, and are not intended to limit the scope of the present disclosure. It would be obvious to a person skilled in the art to which the present disclosure pertains that other modifications based on the technical spirit of the present disclosure in addition to the examples disclosed herein can be implemented.

What is claimed is:

1. A method for designing the composition of a lithium yttrium halide solid electrolyte with a hexagonal close-packed structure, the method being executed by a processor, the method comprising:
calculating possible diffusion paths for lithium ions to migrate an adjacent octahedral site in the a-b plane in each consecutive layer constituting a unit cell with a hexagonal close-packed structure;
calculating the activation barrier energy for lithium ion diffusion for each of the calculated diffusion paths; and
determining the occupancy of yttrium within the unit cell to form a percolation state where diffusion paths with a low calculated activation barrier energy are connected within the unit cell.

2. The method of claim 1, wherein the percolation state includes a Y-1 ordering with one yttrium atom and a Y-free ordering with no yttrium atom in terms of the number of yttrium atoms per unit cell in the ab-plane.

3. The method of claim 1, wherein the calculating of the activation barrier energy is performed by first-principles calculation-based nudged elastic band (NEB) simulation.

4. The method of claim 1, wherein the diffusion paths pass through a tetrahedral site within the unit cell.

5. The method of claim 4, wherein the diffusion paths with a low calculated activation barrier energy include a T_{Y}T_{V} path where one of two octahedral sites adjacent to the tetrahedral site is occupied by yttrium and the other is vacant.

6. The method of claim 5, wherein the diffusion paths with a low calculated activation barrier energy include a T_{V}T_{V} path where both of two octahedral sites adjacent to the tetrahedral site are vacant.

7. The method of claim 6, wherein the T_{Y}T_{V} path has a lower activation barrier energy than the T_{V}T_{V} path.

8. The method of claim 1, wherein the determining of the occupancy comprises:
calculating the interlayer distance between adjacent layers within the unit cell according to the occupancy of yttrium;
calculating the occupancy of yttrium at which the interlayer distance according to the occupancy of yttrium becomes saturated; and
establishing the occupancy of yttrium at which the interlayer distance becomes saturated, as a lower limit of the occupancy of yttrium.

9. The method of claim 8, wherein the calculating of the interlayer distance according to the occupancy of yttrium is performed by first-principles calculation simulation.

10. The method of claim 1, wherein the determining of the occupancy comprises establishing the maximum value of the occupancy of yttrium for formation of the percolation state as an upper limit of the occupancy of yttrium.

11. The method of claim 1, further comprising: after the determining of the occupancy, calculating the composition of the lithium yttrium halide on the basis of the occupancy of yttrium.

12. A computer-readable storage medium storing instructions configured to cause, when executed by at least one processor, to implement predetermined operations by the at least one processor, wherein the operations comprise:
calculating possible diffusion paths for lithium ions to migrate an adjacent octahedral site in the a-b plane in each consecutive layer constituting a unit cell with a hexagonal close-packed structure in a lithium yttrium halide solid electrolyte;
calculating the activation barrier energy for lithium ion diffusion for each of the calculated diffusion paths; and
determining the occupancy of yttrium within the unit cell to form a percolation state where diffusion paths with a low calculated activation barrier energy are connected within the unit cell.

13. The computer-readable storage medium of claim 12, wherein the percolation state includes a Y-1 ordering with one yttrium atom and a Y-free ordering with no yttrium atom in terms of the number of yttrium atoms per unit cell in the ab-plane.

14. The computer-readable storage medium of claim 12, wherein the calculating of the activation barrier energy is performed by first-principles calculation-based nudged elastic band (NEB) simulation.

15. The computer-readable storage medium of claim 12, wherein the diffusion paths pass through a tetrahedral site within the unit cell.

16. The computer-readable storage medium of claim 15, wherein the diffusion paths with a low calculated activation barrier energy include a T_{Y}T_{V} path where one of two octahedral sites adjacent to the tetrahedral site is occupied by yttrium and the other is vacant.

17. The computer-readable storage medium of claim 16, wherein the diffusion paths with a low calculated activation barrier energy include a T_{V}T_{V} path where both of two octahedral sites adjacent to the tetrahedral site are vacant.

18. The computer-readable storage medium of claim 17, wherein the T_{Y}T_{V} path has a lower activation barrier energy than the T_{V}T_{V} path.

19. The computer-readable storage medium of claim 12, wherein the determining of the occupancy comprises:
calculating the interlayer distance between adjacent layers within the unit cell according to the occupancy of yttrium;
calculating the occupancy of yttrium at which the interlayer distance according to the occupancy of yttrium becomes saturated; and
establishing the occupancy of yttrium at which the interlayer distance becomes saturated, as a lower limit of the occupancy of yttrium.

20. The computer-readable storage medium of claim 10, wherein the calculating of the interlayer distance according to the occupancy of yttrium is performed by first-principles calculation simulation.
